(19) United States
(12) Patent Application Publication
(10) Pub. No.: US 2009/0011457 A1
(43) Pub. Date: Jan. 8, 2009

(54) Method and device for testing the bactericidal effect of substances

(76) Inventors: Daniel Faeh, Teufen (CH); Caroline Amberg, Teufen (CH)

Correspondence Address: MOETTELI & ASSOCIATES SARL, ST. LEONHARDSTRASSE 4, ST. GALLEN, CH-9000, CH

(21) Appl. No.: 11/547,726

(22) PCT Filed: Apr. 6, 2005
(86) PCT No.: PCT/CH05/00197

§ 371 Date: Sep. 23, 2008

(30) Foreign Application Priority Data
Apr. 6, 2004 (CH) 599/04

(51) Int. Cl. C12Q 1/18

(52) U.S. Cl. 435/32

(57) ABSTRACT

The method for checking the degree of microbial loading of laundry after a wash cycle is characterized in that a defined amount of living microorganisms are washed together with the laundry, in that the number of microorganisms still living after the end of the wash process is determined and in that the effectiveness or quality of the wash process is determined from the difference between the original amount of living microorganisms and the amount of microorganisms which still remain alive after the wash process. A device for carrying out this method comprises inter alia a container (90) in which the microorganisms are held so that the wash liquid can enter this container (90) but the microorganisms cannot leave this container.

The present invention relates to a method for testing the bactericidal effect of substances and to a device for carrying out this method.

Methods of this species can be used for testing the bactericidal effect for example of disinfectants, antiseptics, detergents, detergent components etc. A method of said species has been published by Univ.-Prof. Dr. Walter Koller, Vienna, in Zbl. Bakt. Hyg., I. Abt. Orig. B176, 463-471 (1982). Germ carriers are used in this method. These are contaminated batiste cloths, which are enclosed between two membrane filters. The system acts as a perfusion chamber. Although the perfusion chamber allows water and the active agents dissolved in it to cross into the interior of the system, it nevertheless prevents bacteria emerging from the system. After conclusion of the wash process, the germs still living in the system are counted. This counting can only be carried out with means which are available exclusively in laboratories equipped for this. The results of such studies with the conventional plate counting method are available only in one to two days, which represents too long a time span for practical purposes. Although the counting can be accelerated with the most recent analysis equipment, the provision of such equipment nevertheless requires great investment.

It is an object of the invention to provide a method and a device for carrying out this method, which make it possible to obtain information about the degree of microbial loading of laundry, or information about the degree of bacterial contamination (=hygiene status) of laundry after a wash cycle in a washing machine, when wash temperatures in the range of 30 to 60° C. are employed. The method and the device should furthermore be configured so that semiquantitative information can also be obtained about the quality of the wash process. At the same time, the method and the device should be configured so that they can be operated in a straightforward way. The method should also be configured so that the results of such a study are available as promptly as possible. The device for carrying out the method should be configured as a disposable product.

For a method of the species mentioned in the introduction, said objects are achieved according to the invention as defined in the characterizing part of patent claim 1.

Said object is also achieved according to the invention with the aid of a device which is defined in the characterizing part of patent claim 8.

Embodiments of the present invention will be explained in more detail below with reference to the appended drawings, in which:

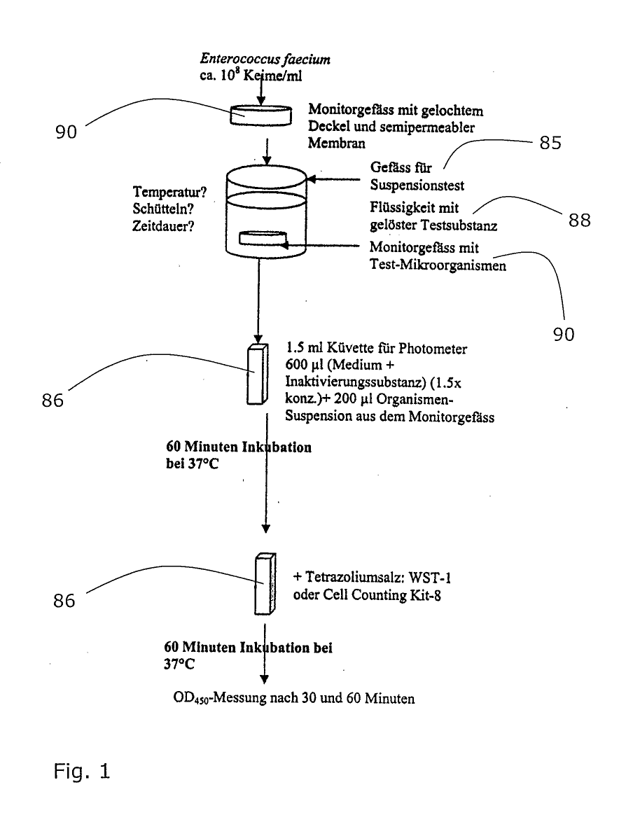

FIG. 1 shows a diagram of the present method,

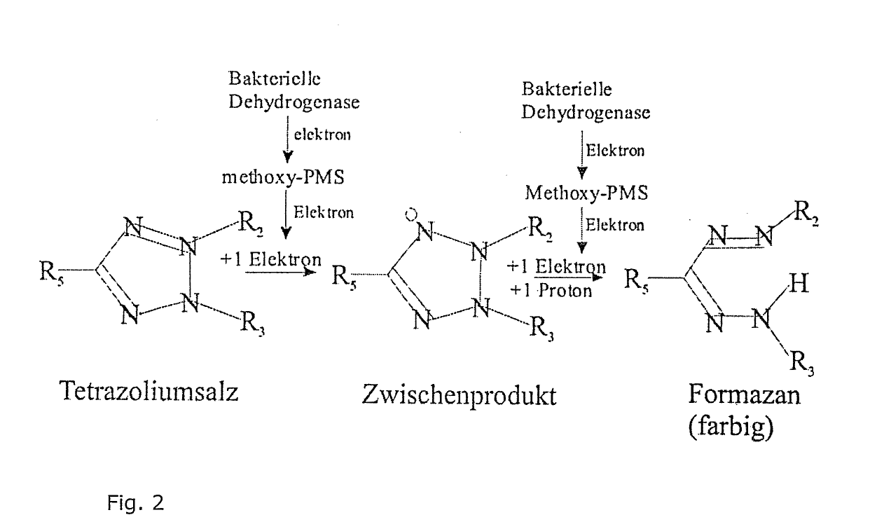

FIG. 2 shows the functionality of some of the substances used in the present method,

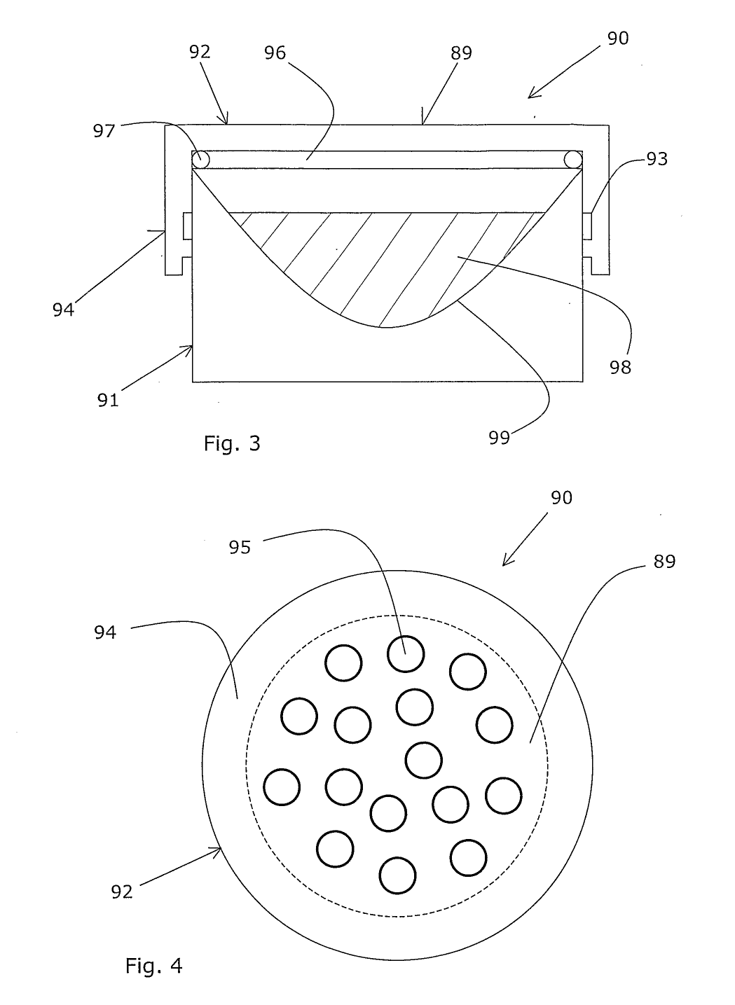

FIG. 3 shows a first embodiment of a monitor vessel in a vertical section, which represents one of the components of a device for carrying out the present method,

FIG. 4 shows the vessel or chamber of FIG. 3 in plan view,

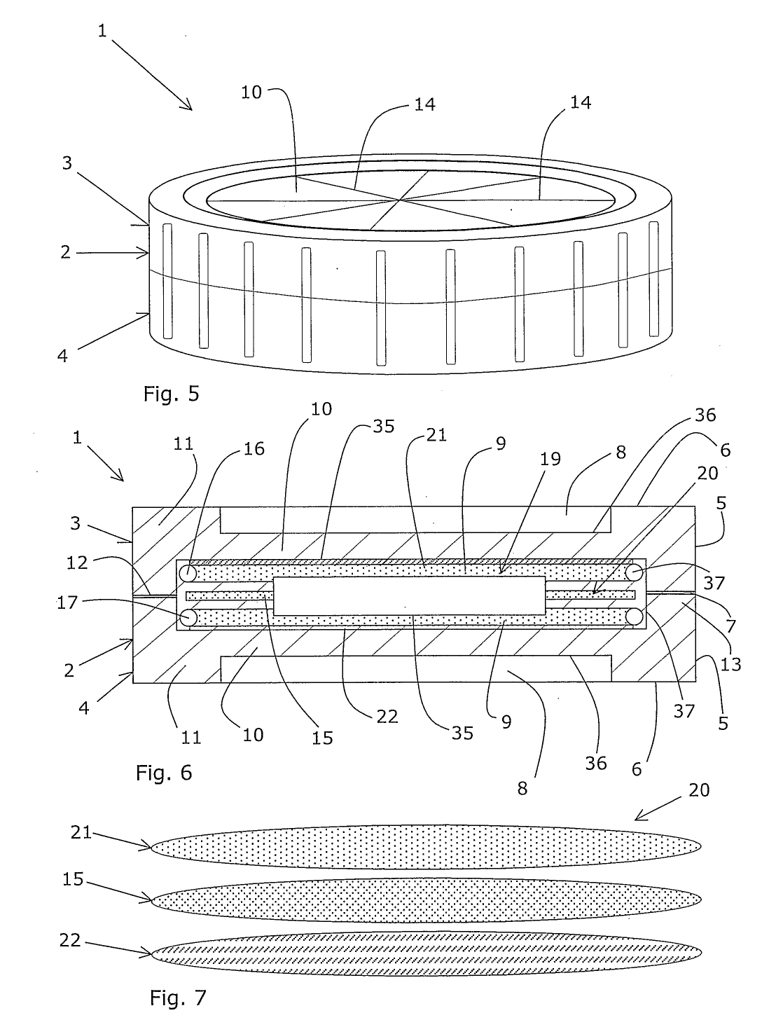

FIG. 5 shows in perspective a second embodiment of the vessel in the state ready for operation,

FIG. 6 shows the vessel of FIG. 5 in a vertical section and only schematically,

FIG. 7 shows the core of the device of FIG. 5 in perspective and schematically,

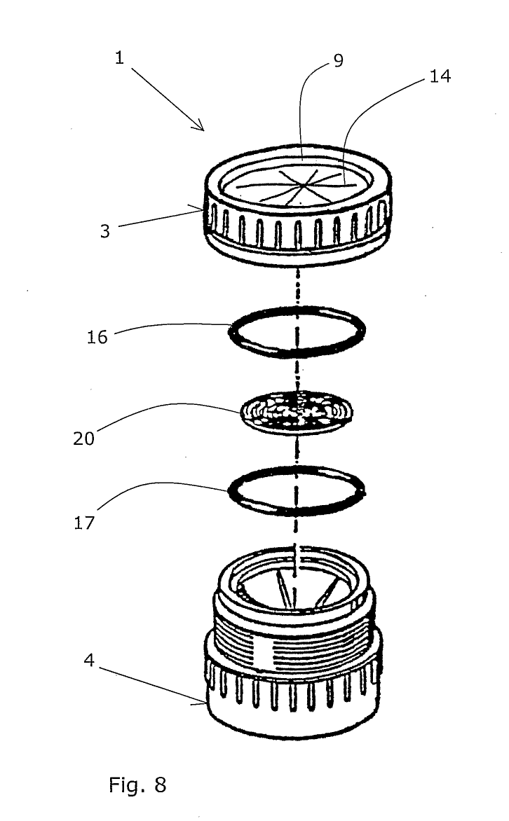

FIG. 8 shows the essential part of the second embodiment of the present device in an exploded representation,

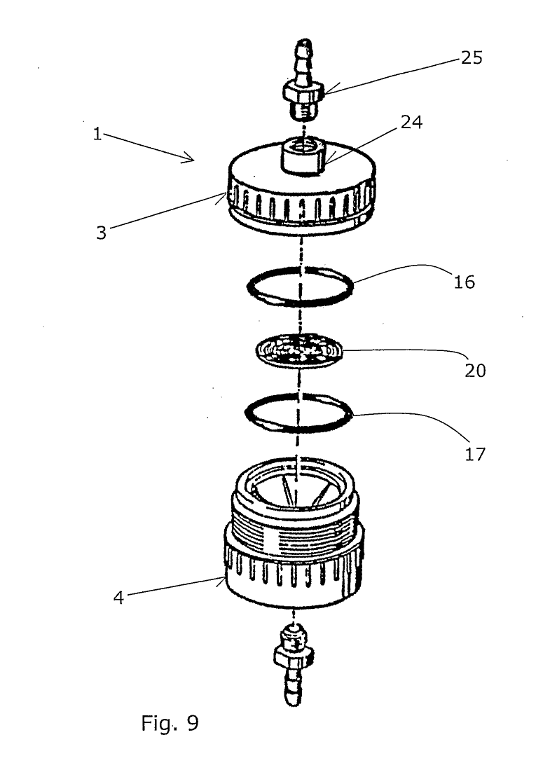

FIG. 9 shows a third embodiment of the present device in an exploded representation,

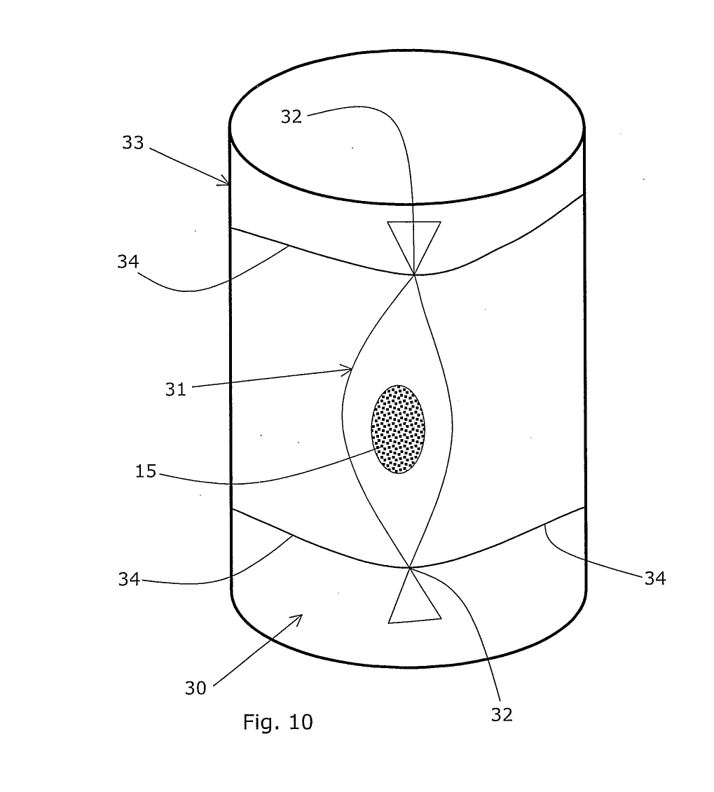

FIG. 10 schematically shows a fourth embodiment of the present device,

FIG. 11 shows the lid of a fifth embodiment of the present device in perspective,

FIG. 12 shows the lower part of the fifth embodiment of the present device in a vertical section,

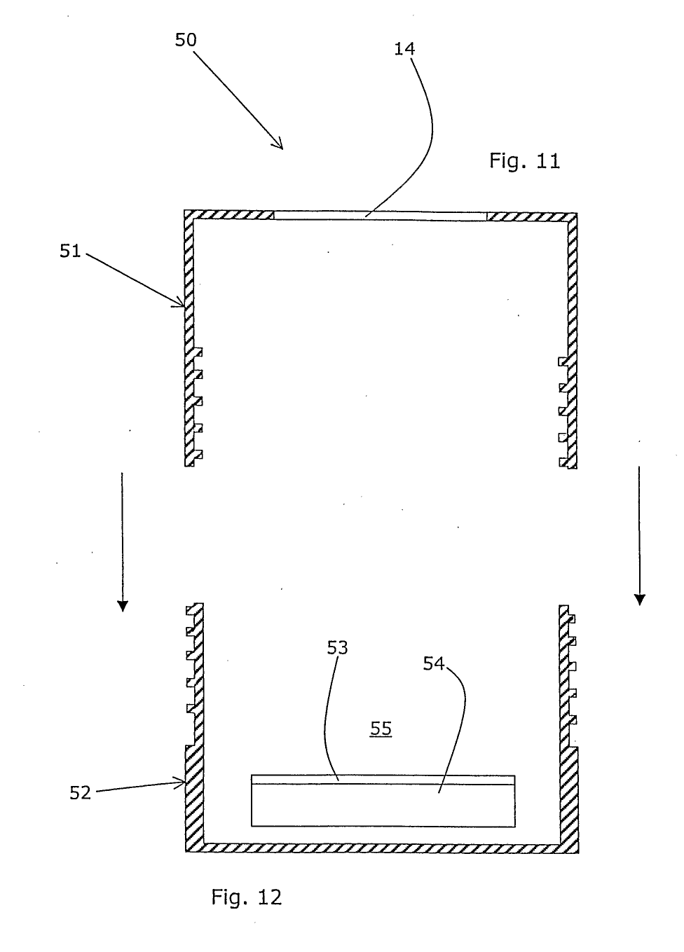

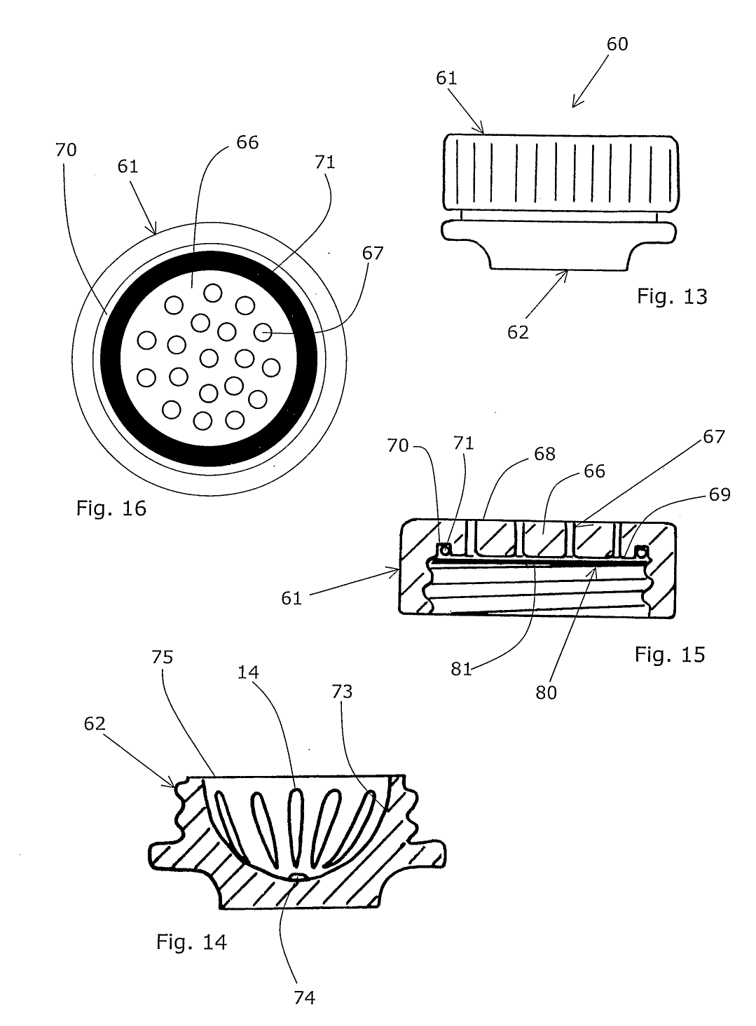

FIG. 13 shows a sixth embodiment of the present device in a side view,

FIG. 14 shows in a vertical section the lower part of the embodiment of the present device as shown in FIG. 13,

FIG. 15 shows in a vertical section the lid of the embodiment of the present device as shown in FIG. 13, and

FIG. 16 shows the lid of FIG. 15 in a view from below, or inside.

FIG. 1 shows a diagram of the procedure of the present method for testing the bactericidal effect of substances 1. In this method, a predetermined or defined number of living microorganisms are exposed for a particular time period to the action of the substance to be tested. The number of microorganisms still living after the end of said action time is determined and the bactericidal effect of the tested substance is deduced from this number of microorganisms still living. Before they are exposed to the action of the substance to be tested, the living microorganisms are put into a liquid with which they form a suspension. This liquid is expediently a nutrient solution or a physiological saline solution. The substance to be tested is expediently provided in the form of a solution or a suspension for the test process. These will also be referred to below as substance liquid 88.

A device for carrying out this method also comprises inter alia a vessel 85 (FIG. 1), in which the test method can be carried out. This test vessel also contains the substance liquid 88. When components of a wash process are intended to be tested, for example, then the wash tub of a washing machine may represent the test vessel 85 and the wash liquor is the substance liquid 88. The present device also comprises a container 90 (FIGS. 1, 3 and 4) which is used to receive the test microorganisms, in particular a defined amount of microorganisms. Such a container 90 may also be referred to as a monitor vessel.

The microorganism container or monitor vessel 90 comprises a hollow lower part 91 and an essentially planar upper part 92. In the example represented in FIGS. 3 and 4, these components 91 and 92 of the container 90 have a circular contour. The microorganisms float in a liquid 98, which lies inside the lower part 91 of the container 90. As already mentioned, this liquid is expediently a physiological saline solution. The interior of the container 90, which is used to receive the test organisms 98, should have a volume of at least 1 ml.

The outer side of the upper edge part of the container lower part 91 is provided with a screw thread 93. The lid 92 comprises a disk-shaped base body 89. A collar 94, which has the shape of a short cylindrical sleeve, hangs down from the edge part of this disk-shaped base body 89 of the container lid 92. The screw thread 93 is likewise formed in the inner surface of this cylindrical sleeve 94, so that the lid 92 can be screwed onto the lower part 91. Holes 95, through which the substance liquid can enter the interior 98 of the container 90 during the interaction between the substance and the microorganisms, are formed in the disk-shaped base body 89 of the lid 92.

The test microorganisms must remain enclosed in the interior 98 of the hollow container lower part 91 during said interaction, so that the results of the test are not vitiated. A semipermeable membrane 96, which covers the entire lower side of the plate-shaped base body 89 of the lid 92 and therefore also the holes 95 in the plate-shaped base body 89 of the lid 92, is assigned to the lower or inner side of the plate-shaped base body 89 of the lid 92. The membrane 96 is configured so that the liquid substance can enter the interior 98 of the monitor vessel 90 during the interaction and interact with the microorganisms. The membrane 96 is however also configured so that it is impermeable for the test microorganisms, so that the microorganisms cannot leave the container 90 through the holes 95 in the lid 92.

So that the test microorganisms cannot leave the cavity in the container lower part 91 through the gap between the lower part 91 and the lid 92 of the container 90, a sealing ring 97 is arranged in that corner part of the lid 92 where the collar 94 meets with the disk-shaped base body 89 of the lid 92. The sealing ring 97 can therefore be compressed between the disk-shaped base body 89 of the lid 92 and the upper edge of the wall of the lower part 91, so that said gap between the threaded parts 93 has a sealing effect for the microorganisms.

It is nevertheless also conceivable for there to be an active unit inside the container 90, which is configured as a perfusion chamber (not shown). This perfusion chamber comprises a carrier for the test microorganisms, which are enclosed in the perfusion chamber. The perfusion chamber furthermore comprises the aforementioned semipermeable membrane 96 comprising pores, which covers the test organisms applied on the carrier.

The following germs, for example, may be used as test microorganisms in the present method:
Escherichia coli,
Candida glabrata,
Enterococcus faecium,
Enterococcus faecalis,
Staphylococcus epidermidis and
Bacillus subtilis.

A defined amount of living microorganisms or living test germs in pure culture, in the case represented in FIG. 1 this is a suspension which contains Enterococcus faecium in an amount of approximately 10⁸ germs, is put into the microorganism suspension 98 is taken from the monitor vessel 90. The microorganism suspension 98 is put into a cuvette 86 (FIG. 1). This may be a cuvette 86 which can be placed in a photometer (not shown) and which for example has a volume of 1.5 ml. The cuvette may contain a liquid medium. This medium is a nutrient solution known per se for the microorganisms.

In a further step of the method, substances which are capable of neutralizing residues e.g. of a detergent in the microorganism suspension 98, or removing them from the suspension 98, may be added to the suspension. Such a substance may also be referred to as an inactivating substance. For example 200 μl of organisms 98 from the monitor vessel 90 may receive approximately 600 μl of liquid, this liquid consisting of the nutrient solution and the inactivating substance. The microorganisms 98 are kept in this liquid for a predetermined time period, for example 1 hour, and at a predetermined temperature, for example 37° C.

After such a pretreatment of the suspension, or after such an incubation time, that section of the present method which leads to the actual evaluation of the quality of the relevant wash cycle can begin. To this end tetrazolium salt is added to the suspension. Living microorganisms contain a bacterial enzyme, dehydrogenase. This enzyme is such that it can reduce the tetrazolium salt to form a colored product, formazan, according to the number of microorganisms still living i.e. according to the activity of the living microorganisms in the suspension. FIG. 2 schematically shows the process in which the tetrazolium salt is reduced to a colored product, formazan, according to the number of microorganisms still living in the suspension 98. The activity of the microorganisms, and therefore also the number of those microorganisms which have survived the interaction process, is deduced from the amount of formazan formed and therefore also from the intensity of the resulting color.

In the next step of the present invention, the optical density or intensity of the light due to the formazan is measured. This measurement may be carried out with the aid of a photometer. The optical density may be measured at a predetermined wavelength, for example 450 nm. Conclusions about the number of microorganisms which have survived the test process, and therefore also the bactericidal effect of the tested substance, can be drawn from the measurement.

The measurement of the optical density may nevertheless also be carried out in a more accurate way, specifically on the basis of the increase in the amount of formazan over a particular time period (not shown). The microorganisms present in the suspension are firstly incubated for a first time period, for example 30 minutes, and at a particular temperature, for example 37°. This is followed by a first measurement of the optical density. The microorganisms present in the suspension are then incubated for a second and subsequent time period, for example 30 minutes, and at a particular temperature, for example 37°. This is followed by a second measurement of the optical density. In this way, the increase in the amount of formazan over a particular time period (for example 30 minutes) can be measured. The increase in the optical density correlates directly with the bacterial activity or number of bacteria. Conclusions about the number of those microorganisms which have survived the interaction process, and therefore also the bactericidal effect of the tested substance, are drawn from the difference between the results of these two measurements.

When testing of wash components is involved then the degree of microbial loading of laundry after a wash cycle, or the hygienic effect of a wash process i.e. the decrease in the number of bacteria during a wash process in a washing machine 85, should be determined. For such tests, the container 90 is configured so that it can be put inside a washing machine 85 without suffering significant damage during the wash process. The container 90 is made of a material which is mechanically very stable, which can be processed mechanically, which is autoclavable (at least up to +121° C./1 atm) and which is washing machine and tumbler proof. The interior of the container should have a volume of at least 1 to 1.5 ml, which is used to receive the sample or test material.

The monitor vessel 90 is filled with a predetermined or defined amount of living test microorganisms 98, this monitor vessel 90 is put into a washing machine 85 together with the laundry and subjected to a wash cycle. The test microorganisms 98 are thereby exposed to the same effects of the wash process as the laundry is. The washing machine 85 may, for example, be a domestic machine or an industrial washing machine. After the end of the wash cycle, the number of microorganisms still living is determined by one of the measurement methods described above. The effect or quality of the wash process, or of the detergent, detergent components or wash method used in the wash process, is deduced from the difference between the defined initial amount of living microorganisms introduced into the wash cycle and the amount of microorganisms still living after the wash cycle. The fewer microorganisms that are still alive after a wash cycle, the more effectively the wash cycle has performed and the lower is the degree of microbial loading of the laundry after a wash cycle, and the greater is the hygienic effect of the wash process.

A second embodiment of the present device is represented in FIGS. 5 to 8. It comprises a container 1, which is designed so that it comprises a hollow base body 2. This container base body 2 may for example have the shape of a cuboid, a cube, a sphere etc. In the case represented, the base body 2 of the container 1 is disk-like. The hollow base body 2 of the container 1 in this embodiment of the present device comprises an upper part 3 and a lower part 4, which may also be referred to as halves 3 and 4 of the container 1. In this embodiment, the base body of each of the container halves 3 and 4 has the shape of a thick disk with a cylindrical lateral surface 5. The base body of each of the container halves 3 and 4 furthermore comprises two mutually opposite disk-shaped large surfaces 6 and 7 lying mutually parallel. A recess 8 or 9 is made in each of the respective large surfaces 6 and 7 of the container halves 3 and 4. The depth of the recesses 8 and 9 in one of the container halves 3 or 4 is less than the thickness of the disk-shaped container halves 3 and 4, specifically so that a partition wall 10 is provided between the bottoms of the recesses 8 and 9 in the respective container halves 3 and 4.

In the circumferential region, each recess 8 and 9 in one of the container halves 3 or 4 is bounded by a ring-shaped edge part 11. This edge part has a side surface 37 lying on the inside. The partition wall 10 is integral with the edge part 11 of the relevant disk 3 or 4. The mutually opposing sections 12 and 13 of the ring-shaped edge part 11 of the container halves 3 and 4, which are hollowed as described above, are provided with a screw thread known per se so that the container halves 3 and 4 can be releasably connected to one another with the aid of this screw thread. This screw thread is preferably configured so that one and a half rotations are sufficient to open or close the housing 2.

Openings 14, which connect the recesses 8 and 9 in the respective disk-shaped container halves 3 and 4 to one another in respect of flow, are made in the partition wall 10 of the relevant disk 3 or 4. In the case represented, these connection openings 14 are configured as slits in the partition wall 10, which extend radially away from the center of the partition wall 10. Besides these slits 14 or as an alternative to them, connection openings 14 with a different shape of their contour may be made in the partition wall 10. The interior of the container 1 is defined by the inwardly lying recesses 9 opening towards one another in the container halves 3 and 4. Said openings 14 connect the interior 9 of the container 1 to its surroundings, in particular to the recesses 8 lying on the outside of the container halves 3 and 4.

The interior 9 of the container 1 should have a volume of at least 1 to 1.5 ml, which is used to receive the sample material. An active unit or perfusion chamber 20 of the present device lies in the interior 9. This perfusion chamber 20 comprises inter alia a carrier 15 for the test microorganisms. This carrier 15 may, for example, be a piece of paper. The carrier 15 may alternatively be configured as a cloth which is made of a textile material, for example cotton. The carrier 15 has the shape of a disk in the case represented, for example made of one of said materials, this disk comprising a circular edge. The diameter of this disk-shaped carrier 15 is dimensioned so that its edge part bears on the essentially cylindrical inner wall 37 of the cavity 9 in the container is 1. An O-ring 16 or 17 respectively lies on either side of the carrier 15 in this edge region 37. The diameter of the cross section of these O-rings 16 and 17 is dimensioned so that the O-rings 16 and 17 are compressed between the carrier 15 for the test microorganisms and the bottom 35 of the inner recess 9 in the container halves 3 and 4, so that no liquid can flow between the cylindrical inner wall 37 of the container cavity 9 and the edge of the carrier 15.

The perfusion chamber 20 of the present invention also comprises disks 21 and 22 (FIG. 3), which are made of a material that is normally used for so-called sterile filters. The pore size of these filters may, for example, be 0.2-0.4 micrometers. Sterile filters normally consist of nitrocellulose and are very brittle. They are therefore exposed to a high damage risk under mechanical loads such as occur during vibrations in the machine. The disks 21 and 22 have essentially the same diameter as the carrier 15, so that they can likewise be accommodated in the interior 9 of the container 1. The filter disks 21 and 22 extend essentially parallel to one another as well as to the carrier 15 for the test microorganisms.

One each of the filter disks 21 and 22 is assigned to one of the large-area sides of the carrier 15 for the test microorganisms. In the case represented in FIG. 6, the respective filter disk 21 or 22 lies between the bottom 35 of the inner recess 9 in the relevant container half 3 or 4 and the O-rings 16 or 17 assigned thereto. In this case one each of the filter disks 21 and 22 is assigned to the inner side or bottom 35 of the respective partition wall 10 of the container halves 3 or 4, and the respective filter disk 21 or 22 thereby covers the openings 14 in the partition wall 10 of the interior 9 of the container 1. It is nevertheless also conceivable for one each of the filter disks 21 and 22 to bear directly on one of the large surfaces of the carrier 15 for the test microorganisms, so that the O-rings 16 and 17 lie between the outside of the relevant filter disk 21 or 22 and the bottom 35 of the relevant inner recess 9.

FIG. 8 shows this first embodiment of the present device in an exploded representation. As explained, this first embodiment of the present of the device is intended to be put in a washing machine.

FIG. 9 shows a third embodiment of the present device. This embodiment of the present device can be used in those cases in which the wash liquor or another liquid can only flow through the present device. On the inside, this third device is essentially configured in the same way as the second embodiment of this device. Instead of the outer depressions or recesses 8 of the second embodiment, the respective container half 3 or 4 of the third embodiment is provided with a spout 24. The respective spout 24 is connected in respect of flow to the interior 9 of the respective container halves 3 or 4. A nipple 25 known per se can be screwed with its threaded end into the respective spout 24. The opposite end part of the nipple 25 is configured for plugging into a tube, through which the liquid can be introduced into the device according to FIG. 9 or discharged from it.

FIG. 10 shows yet another possible embodiment of the present device. The perfusion chamber 30 of this device likewise comprises the aforementioned carrier 15 for the microorganisms. This carrier 15, however, is enclosed in a sleeve 31 which represents a further component of this perfusion chamber 30. The sleeve 31 has essentially the shape of a tube portion. It is made of a material normally used to produce dialysis tubes. In the present case, sections of such a dialysis tube 31 should have pores which are as large as possible. The maximal size of the pores in the tubes 31, which may for example consist of regenerated cellulose, is 50,000 daltons. The fluid exchange thus takes place very much more slowly via such membranes than in the sterile filters described above. If such membrane tubes 31 are used, then the microorganisms may be put on the carrier 15 as a powder or likewise distributed in a liquid.

The tube portion 31 is connected bacteria-tightly in its end regions, and specifically at an expedient distance from the carrier 15. This may, for example, be done with the aid of clamps. Weldable dialysis tubes 31 made of PVDF are also available, although their pore size is 12,000 daltons. They are furthermore not as flexible and cannot withstand such great loads as the cellulose membrane tube 31. The end parts of the tube portion 31, as shown in the case represented, are furthermore closed by tying together the end parts of the tube portion 31 with the aid of a suitable cord 32.

Since the material of the tube 31 has a comparatively low strength, the tube portion 31 is arranged in an open housing 33 made of a stable material. This housing 33 may be configured as a box-like plastic vessel with large slits or holes. In the case represented, the housing 33 is configured as the lateral surface of a cylinder, the end parts of this cylinder surface being open so that the wash liquid can flow through the housing 33. Together with said perfusion chamber 30, which is arranged in this housing 33, this housing 33 is put in a washing machine or the like. Because the bottom regions of the housing 33 are open, the wash liquid can flow through the housing and thereby reach as far as the microorganisms on the carrier 15 in the sleeve 31. In order to prevent the perfusion chamber 30 from slipping out of the housing 33, the perfusion chamber 30 is held inside the housing 33 with the aid of holding means 34 which are known per se. Those sections of the aforementioned cord 32 which extend beyond the sleeve 31 may be used as holding means 34, their ends being fastened on or in the wall of the housing 33.

FIG. 11 represents the lid 51 of a fifth embodiment of the present device 50 in perspective. FIG. 12 shows the lower part 52 of this fourth embodiment of the present device 50 in a vertical section. In vertical section, both the base body of the lid 51 and the base body of the lower part 52 have a U-shaped cross section. The inside of the side wall of the lid 51 and the outside of the side wall of the lower part 52 are provided with mutually corresponding thread halves as described above, so that the lid 51 can be screwed onto the lower part 52. The lid 51 is provided with the openings 14 described above. In the case represented, a semipermeable membrane 53 comprising pores bears on a carrier 54 and this arrangement lies inside the cavity 55 in the lower part 52. The membrane 53 is permeable for water and detergents.

FIG. 3 shows a side view of a fifth embodiment of the present device. This embodiment likewise comprises a lid 61 as well as a lower part 62. Both the lid 61 and the lower part 62 are essentially designed in the same way as the lid 51 and the lower part 52 of the fourth embodiment of this device.

FIG. 14 shows in a vertical section the lower part 62 of the embodiment of the present device shown as in FIG. 13. The inner wall 73 of this lower part 62 has the shape of the surface of a spherical cap. Slit-shaped openings 14, which extend from the central region 74 of the spherical cap bottom part toward the upper edge 75 of the lower part 62, are made in the wall of such a lower part 62. A radially protruding collar 76 is molded onto the outside of the lower part 62, approximately level with the spherical cap bottom 74.

FIGS. 15 and 16 show a further possible embodiment of the lid 61. FIG. 11 represents the lid 61 in a vertical section. FIG. 16 shows the lid 61 in a view from below, or from the inside. Bores 67 extending perpendicularly to the bottom surface 69 and mutually parallel, which are distributed over the surface of the bottom 66, are made in the bottom 66 of the lid 61. These bores 67 extend between the large outer surface 68 and the bottom surface 69 of the lid 61. The bottom surface 69 is planar. A ring-shaped groove 70, in which a seal 71 lies, is made only in the region of the bottom surface 69. This seal 71 may be configured as an O-ring. The diameter of the groove 70 is dimensioned so that the bottom of this groove 70 faces the upper edge 75 of the lower part 62 when the lid 61 is screwed onto the lower part 62. The sealing ring 71 consequently also faces the upper edge of the lower part 62, so that the sealing ring 71 is compressed between the upper edge of the lower part 62 and the bottom of the groove 70.

There is an active unit or perfusion chamber 80 of the present device in the interior 73 of this container 60. This perfusion chamber 80 also comprises inter alia a disk 81, which is made of a material that is normally used for so-called sterile filters. The pore size of this sterile filter may, for example, be 0.2-0.4 micrometers. Sterile filters normally consist of nitrocellulose and are very brittle. They are therefore exposed to a high damage risk under mechanical loads such as occur during vibrations in the machine. Such disks 81 must therefore be put in one of the containers disclosed here when carrying out the present method.

The disk 81 has essentially the same diameter as the interior in the bottom region of the lid 61, so that the disk 81 can be accommodated in the interior of the lid 61. Its edge part bears on the seal 71. After the lid 61 has been screwed onto the container lower part 62, the edge part of this disk 81 is pressed together sealingly between the upper edge 75 of the container lower part 62 and the seal 71. The perfusion chamber 80 furthermore comprises a carrier for the test microorganisms. This carrier may be of one of the types described above. This carrier is applied on the same side of the disk 81 as that facing the interior 73 of the container 60 delimited in the shape of a spherical cap.

The following test germs, which are also prescribed by European Standard No. 1276, are often used in laboratory wash tests:
Staphylococcus aureus,
Escherichia coli,
Pseudomonas aeruginosa and
Enterococcus hirae.

The following test germs, which are not however prescribed by European Standard No. 1276, are often used in laboratory wash tests:
Enterococcus faecalis (Streptococcus faecalis),
Enterococcus faecium,
Candida albicans and
Trichophyton mentagrophytes (dermatophyte).

In the present method, a defined amount of living microorganisms of living test germs in pure culture are put onto the carrier. The test germs to be used may float in a liquid or they may be present in powder form. Drops of the liquid or predetermined amounts of the powder are put onto the carrier. The microorganisms may be fixed on the carrier before introduction into the wash process. This may for example be done with the aid of a gel, e.g. alginate gel. The bacteria are embedded on the piece of fabric in said gel, so that they remain fixed on the carrier. The microorganisms may furthermore be conserved or stabilized before introduction into the wash process, for example dried by freeze drying.

The carrier prepared in this way is closed bacteria-tightly in the perfusion chamber. Microorganisms of only one type are enclosed in the perfusion chamber. The perfusion chamber is leaktight to an extent such that, although the microorganisms cannot escape from the perfusion chamber, the wash liquid can nevertheless come in contact with the microorganisms. If the perfusion chamber lies inside the container, then the microorganisms are enclosed therein. The container is put into a domestic washing machine together with the laundry, and washed with the laundry. If the perfusion chamber 30 is configured as the described tube portion, then this is firstly fastened in the housing 33 and then the housing 33 is put into a domestic washing machine together with the laundry, and washed with the laundry.

After the end of the wash process, the carrier is taken out of the perfusion chamber from the container and the number of microorganisms still living after the end of the wash process is determined. The effect or quality of the wash process is deduced from the difference between the original amount of living microorganisms and the amount of microorganisms still living after the wash process.

During the detection after washing, it is important that only the cells still living should be recorded, because only these can later multiply and because only they can represent a health risk. If a carrier for the microorganisms is used, for example a round piece of fabric, then this carrier is firstly taken out of the cage or container and washed in a physiological solution. The mortality rate can be read directly after the wash process or wash cycle, for example with the aid of a color indicator. This detection can be carried out in a comparatively straightforward way. The mortality rate may alternatively be measured with the aid of the ATP activity. An ATP determination is carried out by direct measurement of the liquid, or by means of swabs in a luminometer.

The number of microorganisms which have survived the wash process may nevertheless also be determined with the aid of so-called gene probes. Gene probes are produced, for example, by VIT-Vermicon. The surviving microorganisms could moreover be measured by other methods, for example impedance (from BioMérieux or SyLab), optoelectrical counting (from FOSS), solid phase cytometry (from ChemScan), ImmunoMagneticSeparation/IMS (from DYNAL).

In principle it would also be possible to put liquid samples on agar plates and incubate them. With this method, however, the results are not available until after 2-3 days. The results of such an evaluation method, on the other hand, are precise. The staff costs for this method are high.

The quality of the wash process is determined on the basis of the ratio between the number of microorganisms which were alive at the start of the wash process and the number of microorganisms which are still alive at the end of the wash process. Such an evaluation may also be carried out with the aid of a program suitable therefor in a computer.

Many manufacturers add substances with a bleaching and/or simultaneously bactericidal action to their detergents, which are intended to effectively combat microorganisms. These manufacturers would like to know at least for themselves whether their additives do actually kill germs during the wash, or to what degree mortality of the germs takes place. The present method and the present devices should make it possible to check the effectiveness of the substances used in the detergent, as well as the effectiveness of the wash process for internal control in the company, in a way which is as simple and rapid as possible. These methods and these devices may be employed by detergent, bleaching and washing machine producers as well as in industrial laundries and in relevant research institutes. The test laundry envisaged may also for example be any textile material which should not be washed at more than 60° C. These are for example non-fast coloreds, wool, silk, synthetic fibers etc. This method and these devices are furthermore suitable for the microbiological evaluation of new products such as machines, programs, detergents etc. as well as for the microbiological evaluation of wash processes within industrial laundries. With minimal preparative work, the present devices can provide sometimes even semiquantitative information about the germ mortality rate in a wash cycle of a washing machine within hours. The devices furthermore allow rapid evaluation of the actual wash process and the corresponding influencing factors such as temperature, time, detergents and mechanical load. It is not necessary to take any samples from the machine during the wash process.

1. A method for testing the bactericidal effect of substances, characterized in that a predetermined or defined number of living microorganisms are exposed for a particular time period to the action of the substance to be tested, in that the number of microorganisms still living after the end of said action time is determined and in that the bactericidal effect of the tested substance is deduced therefrom.

2. The method as claimed in patent claim 1, characterized in that a defined amount of living microorganisms are washed together with the laundry, in that the number of microorganisms still living after the end of the wash process is determined and in that the effectiveness or quality of the wash process is determined from the difference between the original amount of living microorganisms and the amount of microorganisms which still remain alive after the wash process.

3. The method as claimed in patent claim 2, characterized in that a defined amount of microorganisms are put onto a carrier, for example by using drops or in the form of powder, in that the microorganisms are fixed on the carrier, for example with the aid of a gel, before the carrier is introduced into the wash process and/or in that the microorganisms are conserved, for example by freeze drying, before the carrier is introduced into the wash process.

4. The method as claimed in patent claim 1, characterized in that before they are exposed to the action of the substance to be tested, the living microorganisms are put into a liquid with which they form a suspension, in that this liquid may be a nutrient solution, in that the substance to be tested can be introduced into the test process in the form of a solution or a suspension, in that tetrazolium salt is added to the microorganism suspension after the action time has elapsed, in that the tetrazolium salt is reduced by the dehydrogenase enzyme in the test microorganisms according to the number of microorganisms still living, to form a colored product formazan, in that a measurement of the optical density due to formazan is then carried out and in that conclusions are drawn from the result of the measurement about the bactericidal effect of the tested substance and therefore also the number of those test microorganisms which have survived the action time of the tested substance.

5. The method as claimed in patent claim 4, characterized in that conclusions about the number of microorganisms which have survived the action time of the tested substance are drawn on the basis of the increase in the amount of formazan after an incubation time, in that the measurement of the optical density is carried out by incubating the microorganisms present in the suspension or the nutrient solution for a first time period, for example 30 minutes, after which a first measurement of the optical density is carried out, incubating the microorganisms present in the suspension or the nutrient solution for a subsequent second time period, for example 30 minutes, then carrying out a second measurement of the optical density, and drawing conclusions about the bactericidal effect of the tested substance from the difference between the results of the measurements of the optical density.

6. The method as claimed in patent claim 4, characterized in that substances which can remove or neutralize detergent residues are added to the suspension before the tetrazolium salt is added to this suspension, in that the microorganisms are kept in this suspension for a predetermined time period, for example 1 hour, and at a predetermined temperature, for example 37° C. and in that the tetrazolium salt is not added until after this incubation time of the suspension.

7. A device for carrying out the method as claimed in patent claim 1, characterized in that a container, in which the test microorganisms are enclosed, is provided and in that this container is configured so that the wash liquid can come in contact with the microorganisms in the container and so that the microorganisms cannot escape from the container during the wash process.

8. The device as claimed in patent claim 7, characterized in that the container comprises a hollow base body, in that this base body comprises at least one opening which connects the interior of the container to its surroundings, in that the opening is covered by a wall made of a material, in that a defined amount of microorganisms are put onto a carrier and in that this carrier lies inside the container.

9. The device as claimed in patent claim 7, characterized in that the container is essentially disk-shaped and in that at least one opening is made in at least one of the large surfaces of the hollow disk-shaped base body of the container.

10. The device as claimed in patent claim 8, characterized in that at least one opening is respectively made in at least one large surface of the hollow container base body, in that a sterile filter is assigned to the inside of the respective large-surface wall of the container and in that the microorganisms lie between these two sterile filters, which are essentially arranged mutually parallel.

11. The device as claimed in patent claim 7, characterized in that it furthermore comprises a photometer and a cuvette for receiving the suspension which has been taken from the container (90).
